(19) United States
(12) Patent Application Publication
(10) Pub. No.: US 2013/0211410 A1
(43) Pub. Date: Aug. 15, 2013

(54) Patella Resection Instrument Guide Having Optional Patient-Specific Features

(71) Applicant: ConforMIS, Inc., (US)

(72) Inventors: Mark D. Landes, Salem, NH (US); Terrance Wong, Needham, MA (US); Paula Marie Berg, Bolton, MA (US); William Kurtz, Nashville, TN (US); Philipp Lang, Lexington, MA (US)

(73) Assignee: CONFORMIS, INC., Bedford, MA (US)

(21) Appl. No.: 13/762,164

(22) Filed: Feb. 7, 2013

Related U.S. Application Data
(60) Provisional application No. 61/596,222, filed on Feb. 7, 2012.

(51) Int. Cl. A61B 17/17

(52) U.S. Cl. CPC A61B 17/1767
USPC 606/88

(57) ABSTRACT

Disclosed are improved and/or patient-adapted (e.g., patient-specific and/or patient-engineered) patellar clamp systems, surgical methods and related surgical tools. The various clamp assemblies can facilitate one-handed operation by a user and can include modular features that provide for ease of interchangeability to accommodate components having patient specific features that conform or substantially conform to the patient's patellar anatomy.

### RELATED APPLICATIONS

This application claims the benefit of U.S. Provisional Patent Application Ser. No. 61/596,222, entitled “Patella Resection Instrument Guide Having Optional Patient-Specific Features”, filed Feb. 7, 2012, from which priority is claimed under 35 U.S.C. 119, and the disclosure of which is hereby incorporated herein by reference in its entirety.

### TECHNICAL FIELD

The invention relates to improved and/or patient-adapted (e.g., patient-specific and/or patient-engineered) patellar clamp systems and related surgical tools.

### BACKGROUND OF THE INVENTION

When a patient's knee is severely damaged, such as by osteoarthritis, rheumatoid arthritis, or post-traumatic arthritis, it may be desirous to repair and/or replace portions or the entirety of the knee with a total or partial knee replacement implant, which may include the total or partial patellar resurfacing or partial/total excision of the patella. Knee replacement surgery is a well-tolerated and highly successful procedure that can help relieve pain and restore function in injured and/or severely diseased knee joints.

In a typical knee replacement procedure, the surgeon will make an incision through the various skin, fascia, and muscle layers to expose the knee joint with the knee typically flexed at 90 degrees. With the knee relaxed, the surgeon will typically evert or luxate the patella laterally to expose the anterior aspect of the knee joint, and this anatomical structure can be supported by an assistant using a standard operating room tool (i.e., a towel clip or clamp). Where repair and/or replacement is desired, the surgeon may define and/or debride the margins of the patella by removing osteophytes and incising the synovium surrounding the perimeter edge of the patella. The thickness of the native patella can be measured with a caliper with the value recorded or memorized.

Depending upon surgeon preference, and the condition of the patient's existing anatomy, the anterior cruciate ligament may be excised and/or the surgeon may choose to leave the posterior cruciate ligament intact. Various surgical techniques can be used to remove the arthritic joint surfaces, and the tibia and femur can be prepared and/or resected to accept necessary artificial implant components. Both the tibia and femur may receive artificial joint components made of metal alloys, high-grade plastics, and/or any combination to replace native anatomy and desirably function as a new knee joint. In various embodiments, a tibial implant assembly may include a metal receiver tray that can be firmly fixed to the tibia and receive a corresponding plastic insert (also known as a “tibial spacer”). Once the sizing and of the various joint components are completed, the femoral and tibial components are cemented or otherwise secured into place.

In addition to the placement of tibial and femoral artificial joint components, a surgeon may choose to resurface the underside of the patella to attach an artificial articulating surface or “patellar button.” The overall goals of patellar resurfacing include restoration of patellar thickness, restoration of proper patellar tracking, preservation of the extensor mechanism, and restoration of patellar vascularity. Traditionally, however, surgeons have been faced with limited options to resurface the underside of the patella and measure the height of the patella. In many cases, surgeons may choose to “free hand” cut patellar anatomical structures or choose to “make do” with commonly available surgical resecting tools in preparing the surface for an implant.

If a surgeon elects to “free hand” a resection of the patella, there may be various complications that arise due to the impreciseness of the cut, which can include a resulting inability to restore the knee to its normal function. The “free hand” resection technique and planning will be primarily dependent upon the surgeon's experience, skills, his/her assistant's skills, and the surgeon's understanding and “best guess” as to the performance of the chosen surgical tools. Such an approach may involve an intensive, multi-step process that requires multiple measurements (and re-measurements) of the patellar thickness (including measurements of the native patella as well as repeat measurements during resection cuts and after the final resection) and surface preparation to achieve a smooth, symmetrical cut. After a final resection has taken place, the surgeon will often elect to take an additional final patellar measurement using a set of calipers to select the proper patella implant thickness, desirably reconstructing the patella to approximate the native patellar thickness. In addition to thickness measurements, the surgeon will typically use an additional tool, such as a sizing template, to determine the appropriate diameter of the selected patella implant or “patella button.” The sizing template will desirably assist the surgeon in estimating proper component placement to facilitate the ultimate tracking of the patella, and also locate and plan the surgical preparation of the drill holes for the anchoring features of a typical 3-peg “patellar button.” Once the surgical preparation has been completed, the patellar button can be cemented into placed and the resulting patellar tracking can be assessed. Typically, this technique will require the use of one or more assistants, as well as the employment of multiple steps and tools to resurface and position the patella for total or partial knee arthroplasty. In many cases, a miscalculation or other unsuccessful outcome of any of these steps may lead to under-resection, over-resection or oblique undesired cuts to the patella surfaces, causing decreased joint flexion, early implant wear, patellar fracture (i.e., the patella is too thin), instability of the knee, misalignment of the knee resulting in considerably increased forces across the tibio-femoral joint, migration of the implant, improper patellar tracking, and possible failure to alleviate and/or return of knee joint pain.

Even where modular instrument kits may be provided to assist with patellar resection and/or preparation, a variety of the aforementioned issues and complications can still exist. Modular kits typically contain a significant number of disposable and/or non-disposable tools, which could include a variety of patella calipers, clamps, resection tools, drilling templates and sizing templates of various shapes and sizes that could be used to prepare the patella. The sheer number of components involved in such a kit, along with the host of potential tool choices and combinations thereof, significantly increases the amount of back-table space required to accommodate the surgical kit(s), as well as significantly increases the surgeon's difficulty in selecting and/or employing the proper size and shape of instrument. Moreover, even in systems incorporating numerous modular components, the surgeon will still be forced to choose components and/or component combinations that approximate a desired shape and/or size of the target anatomy, and such inaccuracies can result in similar unsuccessful outcomes as those described above. Moreover, additional limitations of modular kits can include (1) a requirement for significant training of surgeons for use of kits involving a significant number of kit pieces and/or component combinations, (2) the opportunity for multi-piece kits to be missing components or other kit pieces that may be improperly shipped to the hospital or other locations, (3) the kit pieces may have improper sizes available, or such pieces may be improperly toleranced, (4) the kit(s) may require significant storage and sterilization resources, and often involve significant “real estate” in the operating room as well as multiple persons to assemble or assist with the surgeon's conduct of the procedure, (5) particularized training and/or skills of the scrub technician may be required, which may limit available personnel for assisting with the surgical procedure, (6) kits may not account for unusual anatomical features, including non-standard and/or unusual patellar shape and/or size, and (7) such kits and the procedures they mandate can significantly increase both preoperative and operative planning and surgical execution time.

As a result, there exists a need in the art for improved patellar clamp assemblies and associated surgical procedures that reduces the number of pieces in a modular surgical kit and/or that facilitates particularization of surgical tools for use by surgeons in conducting patello-femoral resurfacing and/or replacement procedures.

### BRIEF SUMMARY OF THE INVENTION

The present invention discloses novel devices, methods and techniques that can be employed by a surgeon in conducting patellar-femoral resection during partial knee replacement, total knee replacement, knee revision surgery, and any surgery required to repair a damaged or diseased patella or other joint structure. In various alternative embodiments, the features and advantages disclosed herein can be applied with varying utility to surgical procedures for other damaged or diseased articulating joints, such as the ankle, wrist, shoulder, hip, finger, toe and/or vertebrae (i.e. intervertebral discs, costovertebral joints, contravertebral joint and/or facet joints).

A wide variety of embodiments can be constructed in accordance with various teachings herein, including a patellar clamp assembly that includes a clamping body and a plurality of modular clamping heads, one or more of which can be attached to corresponding holding portions of the clamping body. For example, various embodiments can include one or more of the following: (1) Multiple Patellar Head System; (2) Adjustable Patellar Head System; (3) Adjustable and Rotatable Patellar Head System; and (4) Patient-Specific Patellar Head System. In various alternative embodiments, the patellar clamps described herein can integrate measuring features which could include caliper measurement features.

In various preferred embodiments, one or more contact surfaces of the patellar clamp can include an inset, tray, pad or contact component that can be designed to substantially conform to and/or accommodate the contour of the patient's cartilage and/or underlying bone of the patella. The surface features of such component(s) can include patient-specific features derived from anatomical image data taken from pre-operative imaging of the patient using 2D or 3D imaging techniques such as ultrasound, MRI, CT scan, x-ray imaging obtained with x-ray dye and fluoroscopic imaging. Alternatively, or in addition to such patient-specific features, various surface features of such components can include patient-engineered features derived using anatomical image data from the patient in combination with other non-patient data, including database data of average patient measurements and/or modified measurements derived using various engineering formulas.

If desired, the various component surface features could be designed to accommodate concentric or cylindrical patellas, oblong/obround patellas or non-cylindrical patellas. Each of the patellar head engagement systems could optionally contain patellar cut/drill guides or other visible and/or tactile features to assist the surgeon in accurately resecting the patella.

In various preferred embodiments, the patellar clamping devices described herein will desirably accommodate the dimension of the patella, can incorporate patient-specific and/or patient-engineered surfaces or components, and can include drill guides and/or resection guides that may be movable, rotatable and/or re-orientable to facilitate the surgical preparation of the patella.

In various preferred embodiments, the patella clamp will include features that desirably facilitate the one-handed operation of the clamp by the surgeon, thereby allowing the surgeon to hold and/or manipulate the patient anatomy with a first hand, while placing and securing the clamp proximate to the anatomy with the other. Desirably the entire patellar clamp assembly and associated components are easily useable by a single operator, i.e., the surgeon.

In various embodiments, the manufacture of a patellar clamping tool could include the steps of (a) measuring one or more dimensions (e.g., thickness, perimeter, size, or contour) of the intended implantation site or the dimensions of the area surrounding the intended implantation site; and (b) designing a patellar head system or portions thereof incorporating surface features customized for a given patient and/or patient population.

### BRIEF DESCRIPTION OF THE SEVERAL VIEWS OF THE DRAWINGS

FIG. 1 depicts a series of exemplary two-dimensional cross-sectional images of distal femurs and corresponding patellas;

FIG. 2 depicts a plan view of the articulating posterior surface and surrounding anatomy of a patella;

FIG. 3 are schematic views that show various exemplary shapes of patellas and trochlear grooves of associated femurs;

FIG. 4A depicts a side view of one embodiment of a patellar clamp assembly constructed in accordance with various teachings disclosed herein;

FIG. 4B depicts a side view of an alternative embodiment of a patellar clamp assembly, including a rotatable adjustable-head patellar clamp assembly;

FIG. 5A depicts a side view of an arm assembly of the patellar clamp of FIG. 4A;

FIG. 5B depicts a side view of an arm assembly of the patellar clamp of FIG. 4B;

FIG. 6 depicts a top plan view of the arm assembly of FIG. 5B;

FIG. 7 depicts an isometric view of an actuating lever housing assembly;

FIG. 8 depicts a side view of the actuating lever housing assembly of FIG. 7;

FIG. 9 depicts a top plan view of a bias plate;

FIG. 10 depicts a side view of a bias plate with a longitudinal member;

FIG. 11 depicts a front view of an actuating lever housing assembly;

FIG. 12 depicts an isometric view of one embodiment of a modular patellar head design;

FIG. 13 depicts a bottom view of the patellar head design of FIG. 12;

FIG. 14 depicts a top view of the patellar head design of FIG. 12;

FIG. 15 depicts a front view of an alternative embodiment of a modular patellar head design;

FIG. 16 depicts a side view of the patellar head design of FIG. 15;

FIG. 17 depicts an isometric view of an alternative embodiment of a modular patellar head design;

FIG. 18 depicts a bottom view of the patellar head design of FIG. 17;

FIG. 19 depicts the top view of the patellar head design of FIG. 17;

FIG. 20 depicts the side view of the patellar head design of FIG. 17;

FIG. 21 depicts an isometric view of an alternative embodiment of a modular patellar head design;

FIG. 22 depicts a side view of the patellar head design of FIG. 21;

FIG. 23 depicts a front view of the patellar head design of FIG. 21;

FIG. 24 depicts a front cross-sectional view of the patellar head design of FIG. 21;

FIG. 25 depicts a side view of an adjustable quad-lead screw;

FIG. 26 depicts a top view of the adjustable quad-lead screw of FIG. 25;

FIGS. 27A and 27B depict side views of the head design of FIG. 17 with an adjustable thread screw positioned at 12 mm and 5 mm, respectively;

FIG. 28 depicts one embodiment of a patellar clamp assembly being placed on a luxated patella;

FIG. 29 depicts a side view of the patellar clamp assembly surrounding a patella;

FIG. 30A depicts a side view of a resection axis of a posterior surface of a patella;

FIG. 30B depicts a side view of an angled resection axis of a posterior surface of a patella;

FIG. 31 depicts an isometric view of a patellar head placed on a resected surface of a patella, highlighting the drill guide features;

FIG. 32A depicts an isometric view of a resected patella with drilled peg holes;

FIG. 32B depicts an isometric view of a patellar button being placed on the drilled and resected patella of FIG. 32A;

FIG. 33A depicts a posterior view of the assembled patellar button and resected patella of FIG. 32B;

FIG. 33B depicts a side view of the assembled patellar button and resected patella of FIG. 33A;

FIG. 34 is an isometric view of one alternative embodiment of a patellar clamp constructed in accordance with various teachings disclosed herein;

FIG. 35A depicts a side view of one embodiment of a patellar template for use with the patellar clamp of FIG. 34, with an oblong patellar template;

FIG. 35B depicts a side view of an alternate embodiment of a patellar template for use with patellar clamp of FIG. 34 with an oblong patellar template;

FIG. 35C depicts a partial top plan view of the oblong shaped patellar template of FIG. 35A;

FIG. 36A depicts a side view of a reamer assembly for use with the embodiment in FIG. 34;

FIG. 36B depicts a side view of an alternate reamer assembly for use with the embodiment in FIG. 34;

FIG. 37 depicts a side view of a patellar template secured to an arm of the patellar hub;

FIG. 38 illustrates a partial view of a patellar hub and baseplate, with an associated patient-specific insert;

FIGS. 39A through 39D depict various drill hole patterns that can be achieved using various features of the patellar clamp of FIG. 34;

FIG. 40A depicts a partial side view of a reamed patella that could accept an oblong inlay patellar button;

FIG. 40B depicts a top view of a reamed patella with an inlay depth;

FIGS. 41A through 41C depicts a top view of a patella being resected using the embodiment of FIG. 34;

FIGS. 41D and 41E depict top plan views of one embodiment of a reaming plug and associated resection view using the embodiment of FIG. 34;

FIG. 42 depicts top plan views of a standard sized button and an oblong shaped button on respective oblong shaped patellas; and

FIG. 43 depicts a top plan view of a resected oblong patella with an associated oblong shaped patellar button.

### DETAILED DESCRIPTION OF THE INVENTION

Imaging Techniques

Various features of the present invention may include the employment of a variety of imaging techniques that are suitable for measuring thickness, size, area, volume, width, perimeter and/or surface contours of the diseased patella. Such imaging techniques may be desirable to recreate natural or substantially similar natural surfaces and/or electronic image data thereof, facilitating the design and/or derivation of the specific patellar assembly to repair or replace the patella during surgery. Using the proper patellar assembly with specifically designed shaped and/or contoured patellar heads may significantly improve alignment of the tool and/or other surgical tools, thereby improving alignment of patellar features with the articular or implant surfaces and resulting joint congruity because of more accurate resection and placement of the patellar button. Poor alignment and poor joint congruity can, for example, lead to instability of the joint. In the knee joint, instability typically manifests as a lateral instability of the joint, although a wide variety of manifestations, including patient pain and/or inflammation, can result.

Various imaging techniques contemplated herein can include conventional methods of x-ray imaging and processing, x-ray tomosynthesis, ultrasound including A-scan, B-scan and C-scan, computed tomography (CT scan), magnetic resonance imaging (MRI), optical coherence tomography, single photon emission tomography (SPECT) and positron emission tomography (PET), T1 and T2-weighted spin-echo imaging, gradient recalled echo (GRE) imaging, magnetization transfer contrast (MTC) imaging, fast spin-echo (FSE) imaging, contrast enhanced imaging, rapid acquisition relaxation enhancement (RARE) imaging, gradient echo acquisition in the steady state (GRASS), and driven equilibrium Fourier transform (DEFT) imaging, among other imaging techniques and methods well known in the art. Such imaging can be employed to obtain information on the patient's patella and/or trochlear groove, as well as other bony and/or soft tissue structures such as adjacent bone structures, ligaments and tendons, etc., which can be used to derive and/or select a proper patellar tool assembly and/or patellar head components.

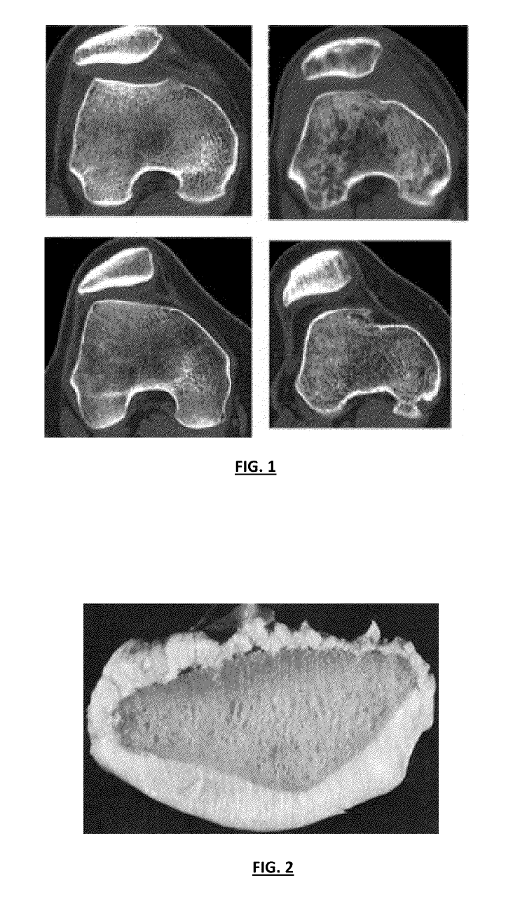

FIGS. 1 and 3 depict exemplary cross-sectional views of a variety of shapes of a patient's patella and associated trochlear articulating surface, and such detail may be captured to properly obtain measurement information on the patella and/or other adjacent anatomical structures.

FIG. 2 depicts a top plan view of a patella and surrounding joint structures, which can include a relatively standard shape. Such images and/or image data can desirably provide highly accurate dimensions and surface/subsurface feature measurements that can assist with the manufacture and/or selection of a patient-specific patella head design. In various embodiments, such measurements can be collected for storage, aggregation and/or classification in a database library.

In various embodiments, anatomical dimensions and/or measurements can be based on three-dimensional (3D) images or two dimensional (2D) images, or sets of two-dimensional images ultimately yielding 3D information. Two-dimensional and three-dimensional images, or maps, of the patella alone, the trochlear groove alone, the combination of the patella and trochlear groove and/or any such data in combination with a movement pattern of the joint, e.g. flexion-extension, translation and/or rotation, can be obtained. 2D images can include information on movement patterns, contact points, contact zones of two or more opposing articular surfaces, and movement of the contact point or zone(s) during joint motion. In addition, imaging techniques can be compared over time, for example to provide up-to-date information on the shape and type of material needed.

In one exemplary embodiment, a surgeon or designer may use measurements of thickness, size, area, volume, width, perimeter and/or surface contour data of the diseased patella obtained from a reference population or from a database library, where the data collected from the reference population may be stored in a database which can be periodically or continuously updated. The patella assembly or the patella heads (i.e., one or both of the superior or inferior clamp surfaces) can be derived and/or selected using the captured measurements from the referenced population or various patient-specific or patient-engineered measurements can be correlated to the reference population database to predict measurements, shapes or contours that may be necessary for optimal resection of the patella during surgery. Alternatively, the measurements of the patella for a reference population or database library can be used to design a pre-made, “off-the-shelf” patella head or patellar assembly closely matching at least one or more of these measurements. In one alternative embodiment, approximate “blanks” for such tools and devices can be designed, manufactured and warehoused using such population data, with the blanks subsequently processed to a more exacting size and/or shape using patient-specific data at a future time when desired.

Patellar Clamp Assemblies

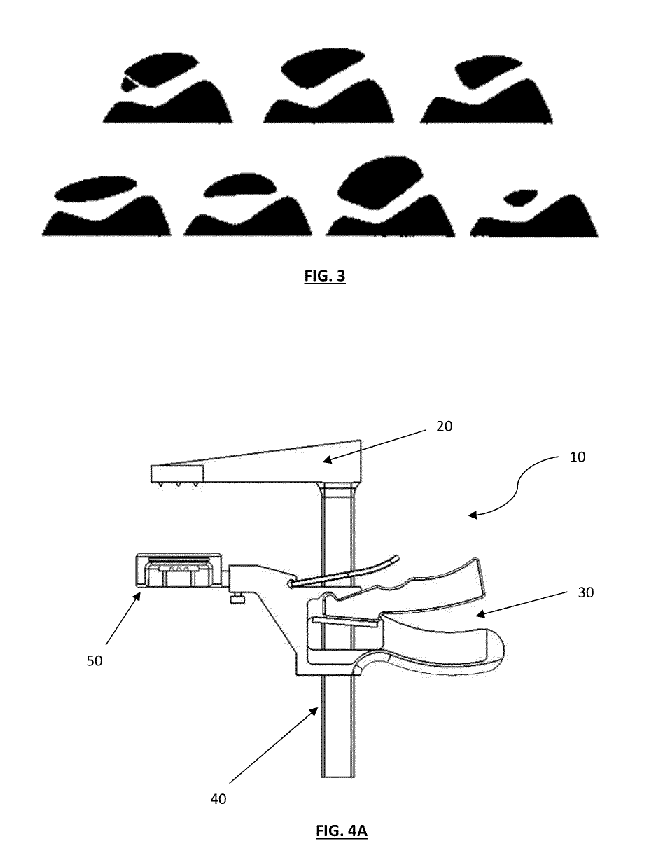

In one preferred embodiment, such as shown in FIG. 4A, a patella clamp assembly may be designed as a sliding clamp that allows for a one-handed operation, with a fixed component having a first jaw feature and a sliding component having a second jaw feature. The device may be fitted with a trigger grip handle, and a quick release lever mechanism. By squeezing the trigger grip handle multiple times, the sliding jaw can be advanced relative to the fixed jaw bar until a desired orientation of the jaws is obtained, and a preference of a light or significant pressure exerted on one or more objects between the jaws is obtained. When compression is no longer desired, the quick release lever mechanism may be pressed or otherwise activated to release the bias plate (and thereby release the pressure) for removal of the device and/or to allow re-adjustment of the pressure and/or to re-measure patellar thickness. In various alternative embodiments, the sliding jaw may be designed or assembled as a spreader or retractor, or the device may include both retraction and/or compressive features. If desired, the sliding jaw may be disassembled by pressing the bias plate, and subsequently inserting the sliding jaw upside down into the assembly, thereby causing the assembly to act like a retractor. The trigger grip handle in such an embodiment can be squeezed to allow the sliding jaw to advance away from the fixed jaw, which can be used to push apart or spread skin, materials, muscle, etc.

FIG. 4A depicts one embodiment of a patellar clamp assembly 10 comprising a multi-piece assembly that may be used for all types of surgeries, including resection of the patella, as well as for a variety of operative functions. The patellar clamp assembly 10 includes a longitudinal rod or member 40 with an arm 20, an actuating lever housing 30, and a fixed patellar head system 50.

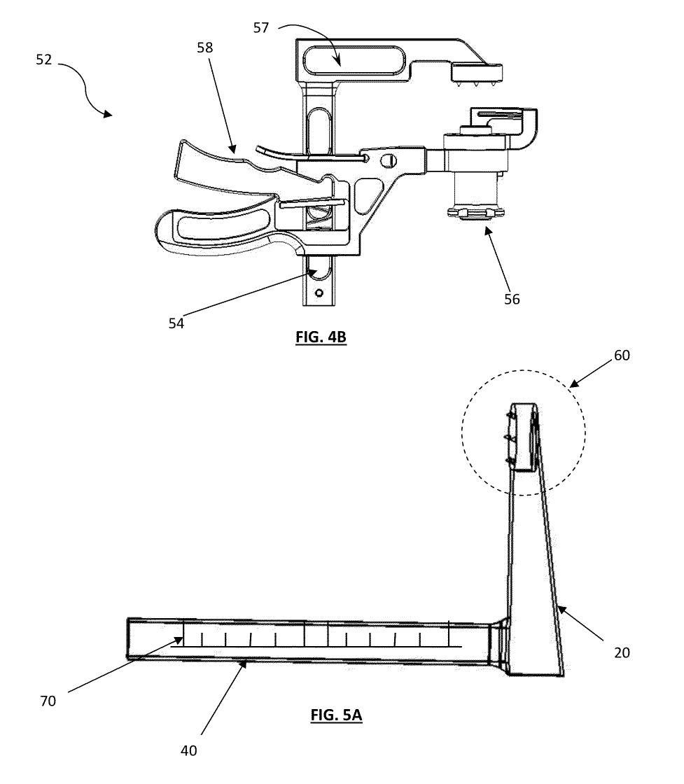

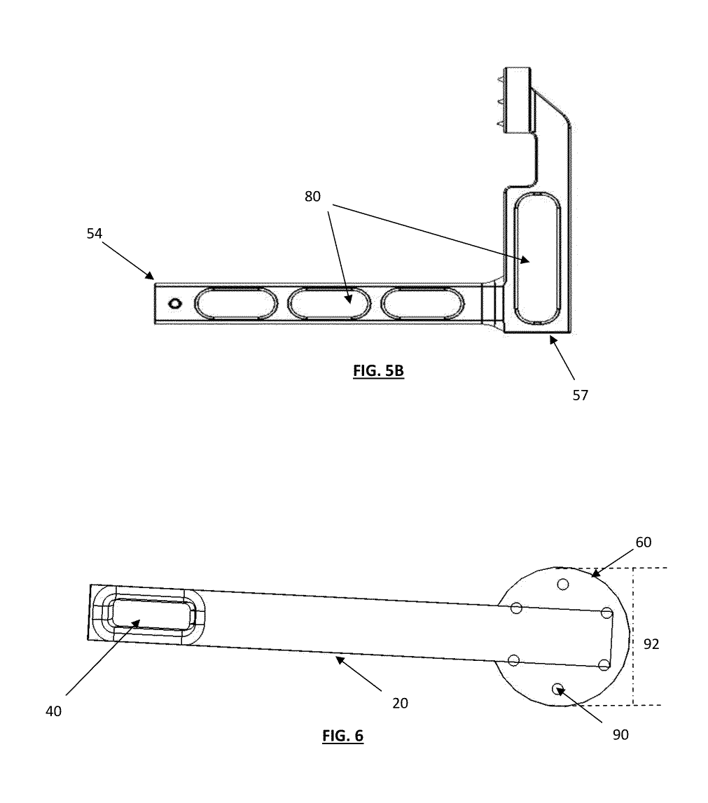

The longitudinal member 40 can be designed in a variety of shapes and/or sizes, and can integrate various operative functions. In one embodiment, the longitudinal rod or member 40 can be designed as a solid rod that is rectangular in shape. Alternatively, the longitudinal member 40 may be formed in other shapes, such as round, triangular, square, or various other shapes that may assist with the actuation and/or translation of longitudinal member 40 within the actuating lever housing 30. As best seen in FIG. 5A, the longitudinal member 40 may incorporate a measuring feature or caliper measurement integrated along its length to measure separation between the jaws, which may reflect a desired or measured patella thickness. Caliper measurements integrated into the longitudinal rod may have various graduations 70 to match the native patellar thickness and/or the resected thickness measurements. In one embodiment, such graduations 70 may be scaled from 0 mm to 50 mm, although a wide variety of such graduations, such as 0 mm to 12 mm or 0 mm to 100 mm, may be used (depending upon the scale of the relevant anatomy to be measured). In various alternative embodiments, the graduations 70 may indicate a desired resection depth, or may reflect appropriate implant choices from a plurality of implants in a kit, depending upon depth of resection, etc.

As depicted in FIGS. 5A, 5B and 6, the longitudinal member 40 may include a fixed jaw and/or arm 20. The arm 20 may connect to a patellar platform or patellar contact surface 60. The patellar platform or contact surface 60 can be designed to accommodate one or more standard sized patellas or it may be designed to reflect one or more patient-specific dimensions 92 (see FIG. 6). The patellar platform may include a variety of patellar gripping or securing features 90, such as spikes, extending protrusions or pegs. The various securing features 90 may be arranged around the circumference of the patellar platform or in any other arrangement to help grip the posterior side of the patella. In various alternative embodiments, the platform 60 may be designed and/or selected to include patient-specific, patient-adapted and/or patient-engineered features that conform to or otherwise accommodate a specific patient's anatomy.

The longitudinal member 40, the arm 20, and the patellar platforms or patellar contact surfaces 60 may be formed from a wide variety of biomedical and/or biocompatible materials, including materials that exhibit superior properties for their intended use, such as high performance polyethylenes, low friction polymers, titanium, stainless steel, flexible materials or hybrid of biomaterial combinations. The strength, weight, and/or sterilization requirements are desirably considered in designing the longitudinal member 40, the arm 20, and the patellar platforms 60.

FIG. 4B depicts one alternative embodiment of a patellar clamp constructed in accordance with various teachings of the present disclosure. This patella clamp assembly 52 includes cut-outs 54 or other features formed in the longitudinal member, as well as one or more cut-outs 57 formed in the fixed jaw, an actuating lever housing 58 (optionally including cut-outs), and a rotatable-adjustable patellar head system 56. The longitudinal member may be formed into a variety of shapes and integrate various operative functions, and such designs and features will desirably take into consideration various strength, weight, and/or sterilization requirements.

In the present example, the various cut-outs can desirably serve a multiplicity of purposes, which can include reducing the amount of material required for manufacture of the tool, reducing the tool's weight, and even more desirably reducing the “sterilization load” required of the tool as compared to a similar tool design without such cut-outs. The incorporation of cut-outs desirably provide a clear path for cleaning, disinfection and/or sterilization media such as hot water, enzymatic agents, soaps, sterilization steam, dry heat, hydrogen peroxide and/or ethylene-oxide sterilization gas (as well as other physical-contact sterilization media) into and through the tool, including into the various subassemblies that might be difficult or impossible to reach in a standard tool design. By making such areas accessible, the amount of sterilization time and contact requirements can be significantly reduced, and the opportunity for removal of foreign materials and pyrogens is significantly increased.

In various alternative embodiments, the longitudinal member and/or various cut-outs (see 80 of FIG. 5B) may be formed in a variety shapes, such as round, triangular, square, or other shapes for the rod itself or the cut-outs. If desired, the cut-outs can be designed to assist with the actuation or translation of the longitudinal member through the actuating lever housing 58, and such features can include roughened surfaces, detents and/or teeth for interaction with driving elements (not shown). In the disclosed embodiment, incorporating cut-outs into the longitudinal member can significantly reduce the weight of the tool, thereby facilitating the one-handed operation by the surgeon, as well as significantly improve sterilization and sterilizability of the tool and associated components. If desired, cut-outs 80 on the longitudinal member may further include positive stops or springs (not shown) attached or inserted within the cut-outs. Such positive stops could potentially act to require a minimum actuation or translation of the clamp for each squeeze of the actuating lever, or provide some needed measurement or position indication, as desired. A spring (not shown) that may be inserted through, within or attached over the cut-outs 80 may be employed to prevent or inhibit reverse force loading when trying to clamp the patella or prevent the longitudinal member from sliding in reverse and/or from completely disassembling upon release of a bias plate 96 or other feature.

FIG. 7 depicts an isometric view of one embodiment of an actuating lever housing 30. The actuating lever housing may comprised a multi-piece assembly that will desirably induce translation of a patellar head system (i.e., 50 from FIG. 4A or 56 from FIG. 4B) in a desired manner in response to a user's actuation. The actuating lever housing can be designed to include a surface that can grip relevant anatomical features (i.e., a patella) as well as to assist with thickness or other measurements of the patella, or the tool can be employed to retract to pull apart skin, tendons, etc. if the arm is rotated 180 degrees and utilized as a retractor (not shown). The actuating lever housing 30 includes a channel or bore 94 formed in a shape to accommodate the longitudinal member 40. The channel 94 desirably centers the longitudinal member 40 within the channel and allows for longitudinal translational movement of the member along its length, in a direction perpendicular to the channel. In various alternative embodiments, the channel or bore may be positioned such that the actuating lever is parallel to the longitudinal translational movement of the member. In other alternative embodiments, the actuating lever housing may be designed to facilitate rotation and/or relative movement (i.e., a plurality of adjustable positions and/or circumferential movement) in relation to the translational movement of the longitudinal member.

FIG. 8 depicts a side view of actuating lever housing 30. The actuating lever housing incorporates a bias plate 96, one or more actuating levers or handles 100, a patellar head system connection mechanism 120, a locking mechanism 110, a locking or urging plate 115, and optional cut-outs 105.

FIG. 9 depicts a top plan view of the bias plate 96. This bias plate has dual functions, for a locking or wedging function and for a release function. The bias plate 96 shown in this embodiment can be manufactured as a spring-tempered plate having a specific angle 98 and/or shape with a through-hole 122 formed in the plate 96 to assist with locking or wedging. The dimensions and shape of the through-hole 122 are designed to be slightly larger than the cross-section of the longitudinal member, but otherwise substantially matching the shape and approximate dimensions of the longitudinal member's cross-section. This substantial matching facilitates the contact surfaces 124 of the through-hole 122 to “wedge” against the outer walls of the longitudinal member in a known manner when the hole 122 is “tilted” relative to the member, and thereby induce a frictional or wedging force between the member and the bias plate, thereby maintaining the position of the longitudinal member against forces tending to impel the member away from the bias plate 115. However, when a force impels the member towards the bias plate, the bias plate will tilt or “flex” and tend to align perpendicular to the member, which reduces the wedging/friction force and allows the bias plate to slide relative to the member. The angle of the bias plate then resumes its position relative to the member, and desirably “wedges” against the member to resist further forces impelling the member away from the bias plate.

In addition, the bias plate design allows for a user to “release” the locking action by simply depressing the proximal portion of the bias plate towards the handle, which substantially aligns the through-hole with the member and allows relatively free movement of the member relative to the handle. This mechanism may be designed in a variety of alternative ways to allow the locking and release of the longitudinal member during actuation, and designed from a variety of materials, such as metals, plastics, rubbers or a hybrid.

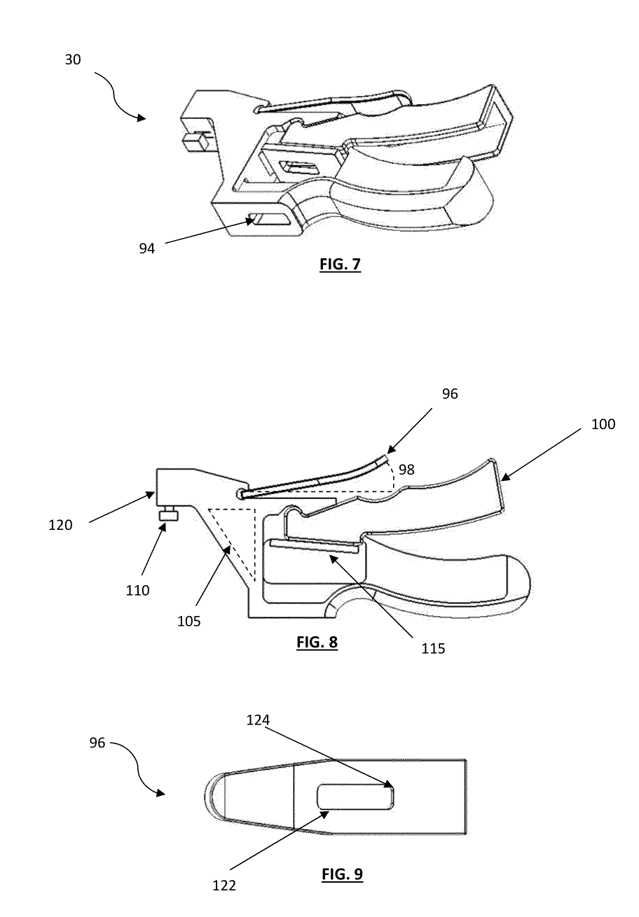

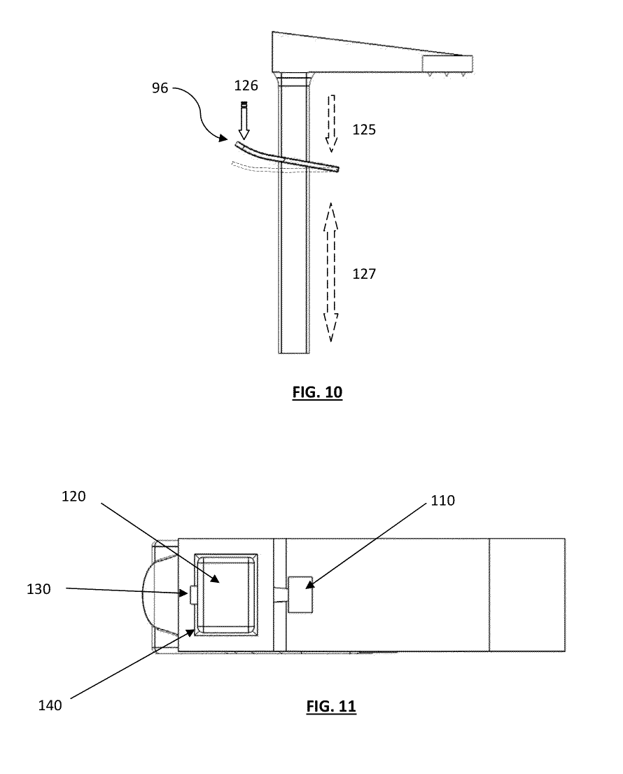

FIG. 10 depicts a side view of a bias plate 96 (shown in an non-engaged position in phantom) with an associated longitudinal member. When the bias plate 96 is not activated, it naturally rests in an upwardly angled orientation. This angled orientation desirably inhibits the longitudinal member's 40 translational motion, biasing such member motion to unidirectional movement 125 through the bias plate. As previously described, the angle of the bias plate desirably wedges the longitudinal arm against movement in the opposing direction. However, if the surgeon chooses to press with a downward force 126 on the bias plate (which disengages the locking mechanism) using his thumb or any finger during one-handed operation, then the through-hole 122 substantially aligns with the longitudinal member, releasing the member from the bias plate 96 and allowing bidirectional movement 127 of the member through the mechanism.

Within the handle, an urging plate 115 (see FIG. 8), positioned proximate to the actuating handle 100, acts in a manner substantially similar to the bias plate 96 (i.e., using wedging and frictional forces to “walk” the member in a desired direction), but in a substantially opposing operation. Essentially, the angle on the urging plate 115 wedges against the longitudinal member 40 when the operator is squeezing the actuating lever or handle 100, pulling the longitudinal member 40 into and through the handle mechanism 30 and advancing the patellar head system toward the patellar platform (i.e., unidirectional movement 125 of FIG. 10). When the squeezing action is completed, release of the handle 100 allows the urging plate 115 to assume a more-perpendicular alignment relative to the longitudinal member 40, thereby allowing the urging plate 115 to slide along the member relatively freely. A spring member (not shown) can be provided to urge the handle to an open position as well as slide the urging plate 115 relative to the longitudinal member 40. During this operation, the wedging action of the bias plate 96 will desirably retain the longitudinal member 40 in its desired position. Once the handle 100 returns to its original position, subsequent squeezing and release of the handle 100 will repeat this operation in a known manner.

In various alternative embodiments, the actuation or the advancement of the patellar head system sliding or moving toward and/or away from the patellar platform could be designed in a variety of other ways, such as using steps to actuate or advance, using a gear with corresponding rack, toothed gear, part or housing to transmit torque or actuation, or may include a motorized or powered actuation with or without programmable maximum compressive forces, a ratcheting mechanism, or any other actuation function or mechanism that allows movement or advancement that is known in the industry.

FIG. 11 depicts a front view of the actuating lever housing, highlighting the patellar head system connection mechanism 120 and a locking mechanism 110. In one embodiment, the patellar head connection mechanism 120 may include a quick release connection. The connection mechanism may contain a central track 130 and have beveled edges 140. The entry of the patellar head connection mechanism can desirably connect to a variety of modular patella heads, which can include various combinations of modular fixed patella head systems, rotatable-adjustable patella head systems, and/or adjustable patella head systems. The various patella head systems may have a central tab 160 (see FIG. 13) that slides into the central track 130 for proper alignment. The central tab 160 may be designed to lock the patellar head systems in place with a “snap” or other audible sound or tactile sensation and/or with a positive stop, as desired. For additional security, the patellar head connection mechanism 120 may include at least one locking stabilizer 110. The locking stabilizer may be designed as a press fit dowel pin, a threaded screw, clips, other types of locking mechanisms, or combinations thereof. The beveled edges 140 on the patellar head connection mechanism 120 desirably provide relatively blunt surfaces, thereby reducing the opportunity for injury to the physician or technician, allowing for atraumatic entry of the patellar head systems while providing proper guidance to connect the patellar head systems. In alternative embodiments, the patellar head connection mechanism could be designed to accept a variety of other useful tools needed during surgery. For example, magnifiers, lights, drills, guides, or other functional tools could replace the patella head systems. In various embodiments, patellar head systems could be dissembled from the patellar head connection mechanism to be replaced with different saw guide depths (offsets) or could be adapted to accommodate spacers to achieve different resection thicknesses. Where desirable, the connection mechanism 120 may be designed as a ball mount, a rotating ball mount or swiveling ball mount to facilitate leveling of the patellar head system, rather than having the patellar head system fixed.

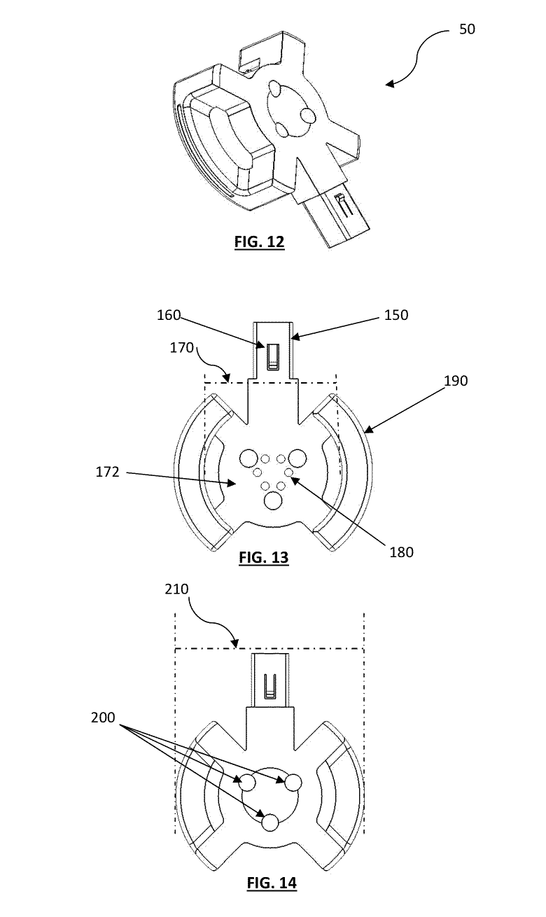

FIG. 12 depicts an isometric view of one embodiment of a patellar head design 50. This design may include a selection of various modular patellar head sizes that can be fixed in width, height, and depth, and may be manufactured in various standard sizes. Alternatively, such fixed patellar head design 50 may be designed and/or selected using patient-specific measurements. The multiple heads of different sizes may be manufactured in virtually any size, including diameters of 29 mm to 44 mm, which can corresponding to different sizes of patellar implants.

FIG. 13 depicts a bottom view of the fixed patellar head embodiment of FIG. 12, highlighting a central tab 160, a connection arm 150, patellar grips or spikes 180, patella cut guides 190, and an anterior contact surface 172 of the patella head embodiment. The connection arm 150 may be designed as a square shape to fit directly into the patellar head connection mechanism 120 (see FIG. 11). Alternatively, the connection arm may be designed in a variety of shapes to achieve easy insertion, guidance and locking prior to use. In addition, the anterior contact surface 172 may have a dimension 170 that is fixed, that is chosen from a library of standard sizes, or may also have dimensions that were derived and/or selected from patient-specific images. The patellar contact surface 172 may also be manufactured flat, at various angles, or contoured to conform to or accommodate the patient-specific shape of the patella. The anterior contact surface 172 may also contain spikes 180 or other features that can be designed to contact and/or extend into the patella, which can include shapes such as smooth pegs, or pointed pegs, or serrated pegs, or pegs with gripping anchors.

In the various embodiment described herein, one of both of the patellar contact surfaces (or portions thereof) can be designed and/or selected to substantially conform to and/or accommodate the contours of various portions of the patient's natural and/or modified anatomical features (if desired), including cartilage, soft tissues, and/or underlying subchondral bone (and various combinations thereof) of the patella.

FIG. 14 depicts a top view of the patellar head design of FIG. 12, highlighting an overall dimension 210 of the tool and drill guides 200. The overall dimension 210 may be derived from standard average patient sizes, chosen from a library of data with specific disease progression, or from patient-specific dimensions. The drill guides 200 may be designed to match the available surgical drill sets available in the operating room or to match a custom surgical drill. In various embodiments, one or more drill guides 200 can be provided on the patellar heads and the placement of the drill guides 200 can be designed and/or selected to match the number of pegs on a standard patella button, on a custom patella button, or on any patient-specific non-standard shape. In one preferred embodiment, the head can be designed with a spherical counter bore on the top side (not shown), such the head can be flipped over and the top side used to contact and clamp the patella button implant component once it is placed on the surgically prepared patellar surface. Such clamping can be especially useful to compress and hold the button to the patella during curing of cement, or can be useful to press-fit the patellar implant to the patella.

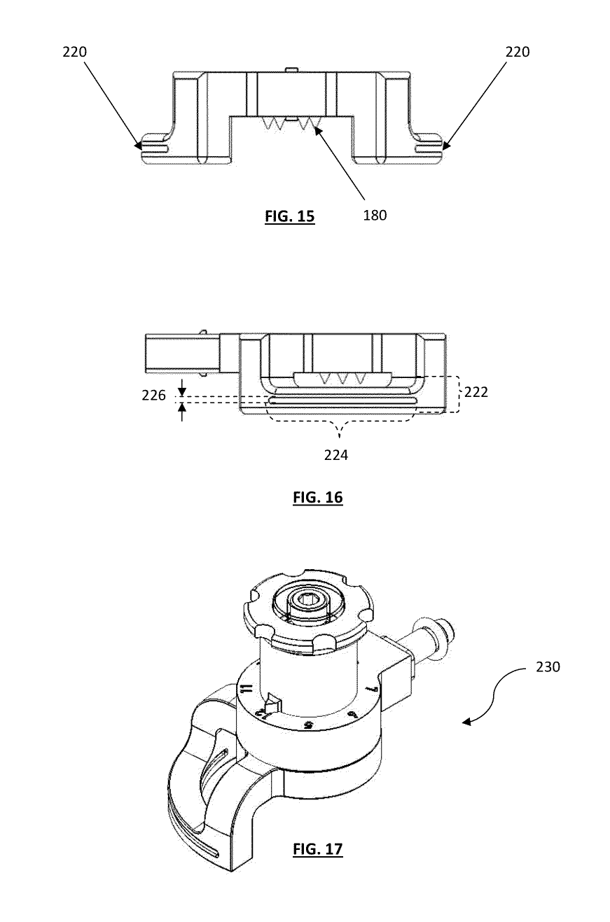

FIG. 15 depicts a front view of the patellar head design of FIG. 12, showing resection guides or cut guides 220 and a series of spikes 180. In various embodiments, the resection guides or cut guides 220 may be placed 180 degrees apart, or may be placed at varying orientations as desired by the surgeon and/or implant designer. Desirably, at least one cut guide 220 will be provided, but several or multiples of such guides may be designed and/or selected for a patellar head.

FIG. 16 depicts a side view of the patellar head design of FIG. 12. This fixed patellar head design may include a set cut guide which includes guide slots, with a resection guide dimension of length 224, width 226, and resection depth 222. These values may be derived in a number of ways, which can include employing standard fixed sizes commonly used for a given patient population, values chosen from a library of data pertaining to the patient's specific diseased state or progression of the disease, or potentially values measured and/or derived using patient specific data and used to create a unique tool for an individual patient based on patient-specific image data.

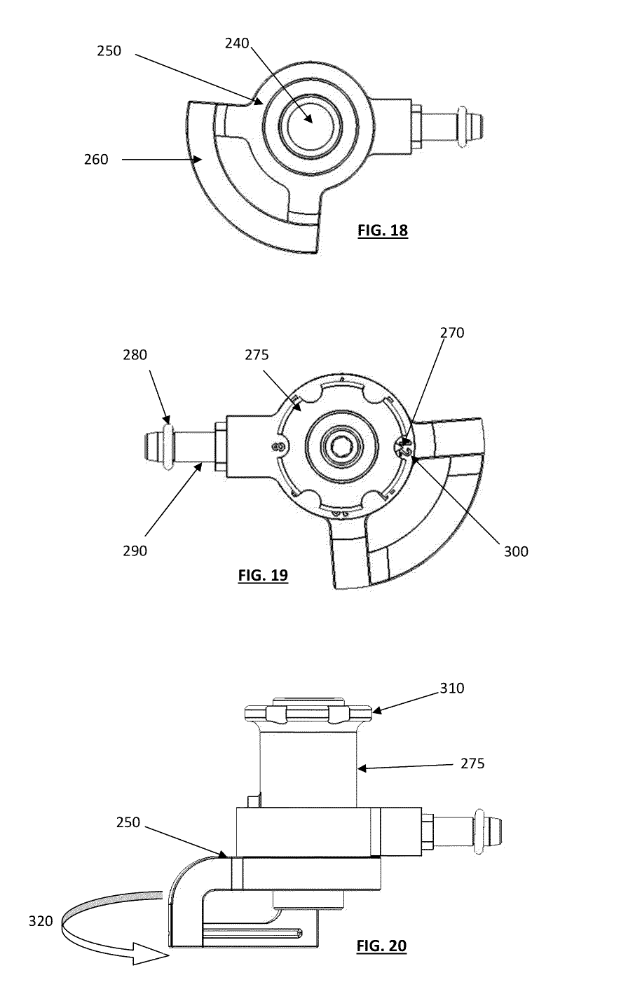

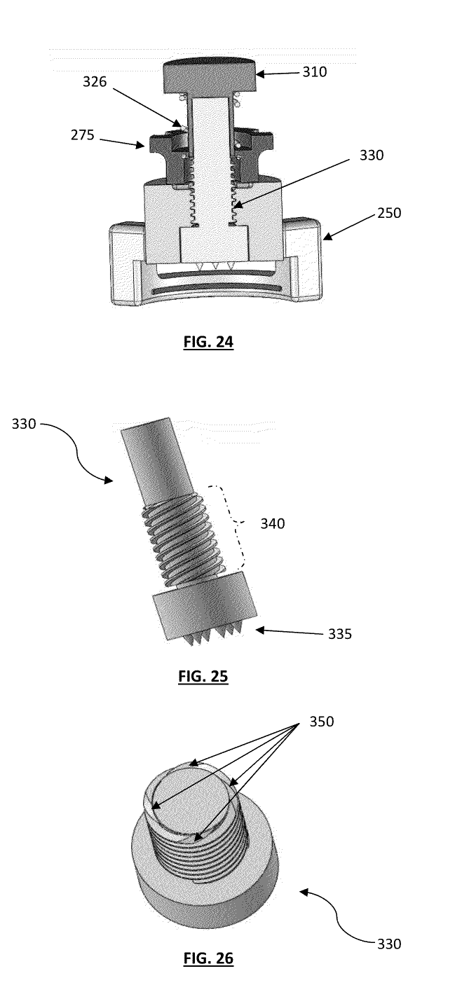

FIG. 17 depicts an isometric view of one alternative embodiment of a rotating-adjustable patellar head design 230. FIG. 18 depicts a bottom view of the rotating-adjustable patellar head design 230 which shows the rotating collar 250, a cut guide 260, and an adjustable patellar platform 240 (see also FIGS. 24 and 25). The rotating collar 250 desirably allows the cut guide 260 to rotate in some portion of or the entirety of 360 degrees around the center axis of the patellar head system, to allow maximum flexibility for the surgeon. The rotating collar 250 may optionally include patient-adapted and/or patient-specific features. For example, some or all of the outer periphery can be patient-specific, matching the outer periphery of the patient's patella. The surface in contact with the patella may also include one or more patient-specific portions, e.g., conforming with the patient's patella surface (and such conforming features can be incorporated into one or both of the opposing contacting surfaces of the inferior and superior jaws of the clamp). Alternatively, in various preferred embodiments, the adjustable patellar platform can be designed with or without spikes 180 (see FIG. 15). If desired, the rotating collar 260 may be removable with a quick release and/or other type of attachment mechanism (not shown).

The cut guides or slots 260 may be configured to accommodate and guide a commonly available and/or customized surgical tool (e.g., a saw or other instrument) for patella resection as previously described. The cut guides or slot 260 dimensions may provide a parallel medial to lateral resection path or accommodate any other angles (i.e. varus/valgus, tilted or anterior/posterior angled designs) for accurate cutting at a desired orientation. A desired resection depth can be regulated by the adjustable patellar platform 240. The adjustable patellar platform can be adjusted and translates to allow adjustment for resection thicknesses, which in one exemplary embodiment may be from 5 mm to 12 mm. In addition, custom cut guides or slots 260 may be designed and/or selected to accommodate a variety of reciprocating saws that are commonly available in surgery operating rooms, or the manufacturer may provide a customized reciprocating saw or other cutting or drilling instrument. If desired, the saw guide dimensions may be designed wide enough to accommodate any standard patella sizes or they may be designed using patient-specific image data, as previously described.

FIG. 19 depicts a top view of the rotating-adjustable patellar head design 230, highlighting an indexing knob 275, resection depth indicators 300, a measured indicator 270, a connection arm 290, and a connection arm seal 280. In one embodiment, the resection depth indicator may include a series of numbers 300 or other indicators designed and/or printed directly onto the patellar head, or directly onto the indexing knob 275. This arrangement can provide quick and easy feedback to the surgeon to understanding the corresponding resection depths for the patient—which in the depicted exemplary embodiment can range from 5 mm to 12 mm. The indexing knob 275 may also incorporate locking or indexing features, such as a ball and detent arrangement, to produce a “snap” or other audible signal, and/or may include resection depth indicator numbers 300 in fractional increments. The indicators may include a specific shape or color, such as a green triangle, so the shape and/or color easily points out the indication number. In various embodiments, the indicator may be manufactured in the form of a slot or window which obscures other values and presents, highlights or otherwise indicates the measured depth indicator 270.

Where desired, the patellar head system can be equipped with a quick release mechanism, which facilitates the connection arm 290 and a connection seal 280 fitting into a patellar head connection mechanism. The connection seal 280 may be manufactured from standard rubber seals or gaskets or may be designed using spring seals, if desired. Spring seals can provide a high tensile force (which will desirably prevent unintended detachment of the patella head from the actuating lever housing) and low compressive force (facilitating modular attachment and/or removal). Other quick release mechanisms used may comprise spring tempered release mechanisms, set screw mechanisms, press fits, snap fits, or other many types of quick release mechanisms known in the industry.

FIG. 20 depicts a side view of the rotating-adjustable patellar head design 230, showing an indexing knob 275 with an ergonomically designed cap or turning knob 310. The turning knob 310 may have a surgeon-specific diameter and/or shape to accommodate the surgeon's hand size or dominant hand (i.e., right or left handed operation), and may integrate other gripping materials (i.e. rubber or texturing features), or may have features that fit the specific fingers used to turn the knob 310 or the index knob 275 in a desired direction 320.

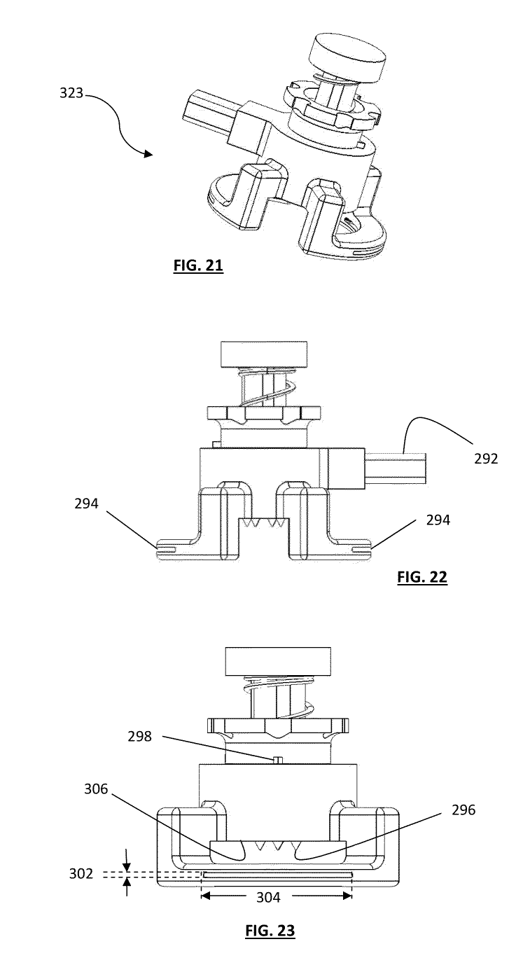

FIG. 21 depicts an isometric view of an adjustable patellar head design 323 with a fixed collar. This adjustable patellar head design 323 encompasses similar features as the rotating-adjustable patellar head design 230, except that the cut guide does not include a rotating collar 250 (see FIGS. 16 and 17).

FIGS. 22 and 23 shows various additional views of the adjustable patellar head design 323. As shown in FIG. 22, the adjustable patellar head design 323 may also include a connection arm 292 and patella cut guides 294. The connection arm 292 may be designed as a square shape to fit directly into the patellar head connection mechanism 120 (see FIG. 11). Alternatively, the connection arm 292 may be designed in a variety of shapes to achieve easy insertion, guidance and locking prior to use. The connection arm 292 may also be designed as a quick lock & release mechanism, such as spring tempered release mechanisms, set screw mechanisms, press fit, snap fit, or other many types of quick release mechanisms known in the industry. In one exemplary embodiment, the patella cut guides 294 may incorporate resection guide surfaces placed 180 degrees apart. Desirably, at least one cut guide 294 will be provided, but several or multiples of such guides may be designed and/or selected for a fixed patellar head.

FIG. 23 is a side view of the patellar head design of FIG. 21, depicting a depth indicator 298, an anterior patellar contact surface 306, anterior contact surface spikes 296 and the length 304 and width 302 dimensions of the cut guide. The cut guide length 304 and width 302 dimensions may be fixed, that is derived or selected from a library of standard sizes, or may include dimensions that were derived and/or selected from patient-specific images to match the patella. The patellar contact surface 306 may also be manufactured flat, at various angles and/or curvatures, or may be contoured to conform to or accommodate the patient-specific shape of the patella. The anterior contact surface 306 may also contain spikes 296 or other features that can be designed to extend into the patella, which can include shapes such as smooth pegs, or pointed pegs, or serrated pegs, or pegs with gripping anchors.

FIG. 24 depicts a front cross-sectional view of an internal assembly of the adjustable patellar head design. The adjustable patellar head includes a turning knob 310, an indexing knob 275, a thread screw 330, a cut guide or slot 250, and a loading spring 326. This arrangement desirably allows adjustment of the resection depth for resection variation. If desired, the knob may include depth indicators, such as 5-12 mm (or other depths), which can be incremented in whole or fractional numbers.

FIGS. 25 and 26 show side and top perspective views of the thread screw 330 of FIG. 24. In the disclosed embodiment, the thread screw 330 includes a quad-lead threaded bolt 340 section that interacts with corresponding thread forms in the head (see FIG. 24) to facilitate patellar resection thickness adjustability. Rotation of the screw desirably raises and/or lowers the spiked patellar platform 335 connected to the turning knob 310. As illustrated in FIGS. 27A and 27B, one embodiment of the thread screw 330 and patellar platform 335 arrangement can be designed to advance 1 mm every 45 degrees of screw rotation, for a varied resection thickness 340 from 5 mm-12 mm, by rotating the turning knob 310 in a desired direction 355. In various alternative embodiments, the lead thread can include single or multiple leads of differing pitches, depending upon the amount of adjustability desired.

In various alternative embodiments, the support of the platform 335 could include a swivel arrangement (not shown) which could allow the platform to swivel relative to other portions of the clamp and desirably accommodate the contour of the patella. If desired, various other surface features could be incorporated into the surface of the platform, in addition to or in place of the spiked features, such as angular or concave cutouts (not shown) on the platform surface to accept and/or accommodate the patellar surface.

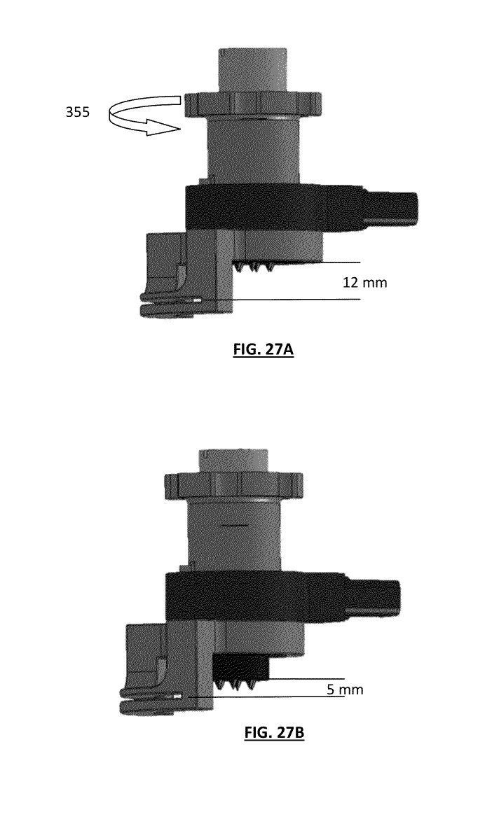

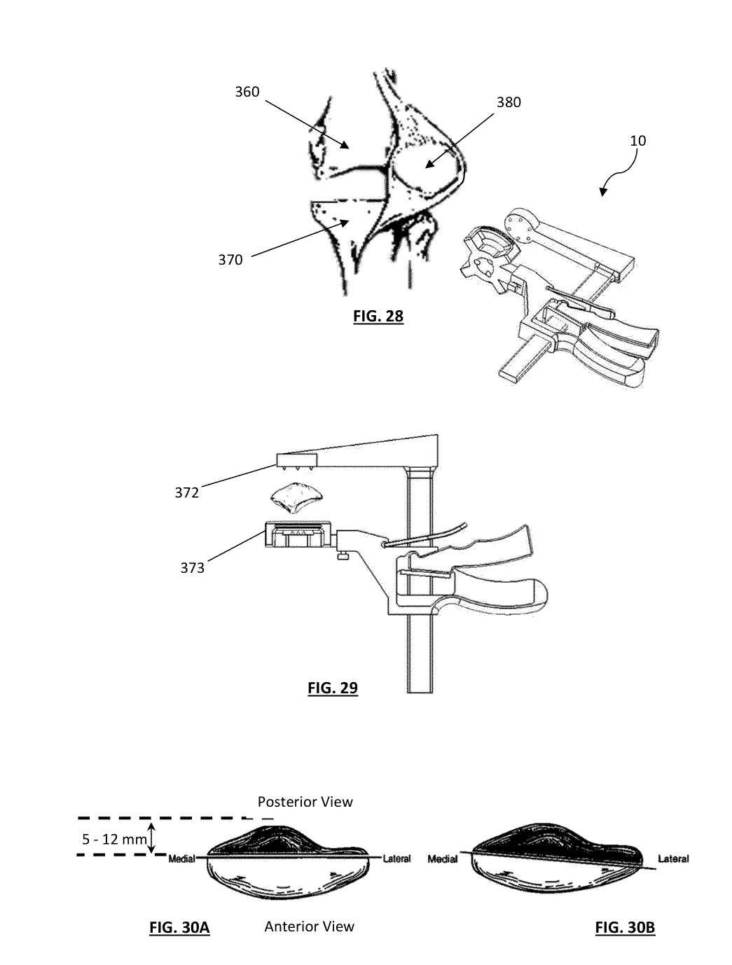

FIG. 28 depicts a patellar clamp assembly 10 approaching an exposed and everted patella 380 and associated femur 360 and tibia 370. As best seen in FIG. 29, the patella can be held and/or manipulated by one hand of the surgeon while the clamp is operated by the surgeon's other hand. The patella will desirably be positioned between the jaws of the clamp assembly, and the jaws subsequently urged to a closed position as previously described. As described herein, one or more of the superior and inferior jaws 372 and 373 of the clamp assembly can include patient-specific, patient-adapted and/or patient engineered features, to accommodate one or more anatomical features of the natural patella and/or resected patellar surface(s). Once the jaws have clamped, encompassed or otherwise secured the patella, one or more resections can be performed on the articulating face of the patella (i.e., the posterior face generally facing the femur), including a flat resection of a desired depth (see FIG. 30A) or a tilted or angled resection (see FIG. 30B), as desired. In various alternative embodiments, the resection may include a multi-level and/or multi-angled resection, potentially separated by a bevel or chamfer cut region (not shown).

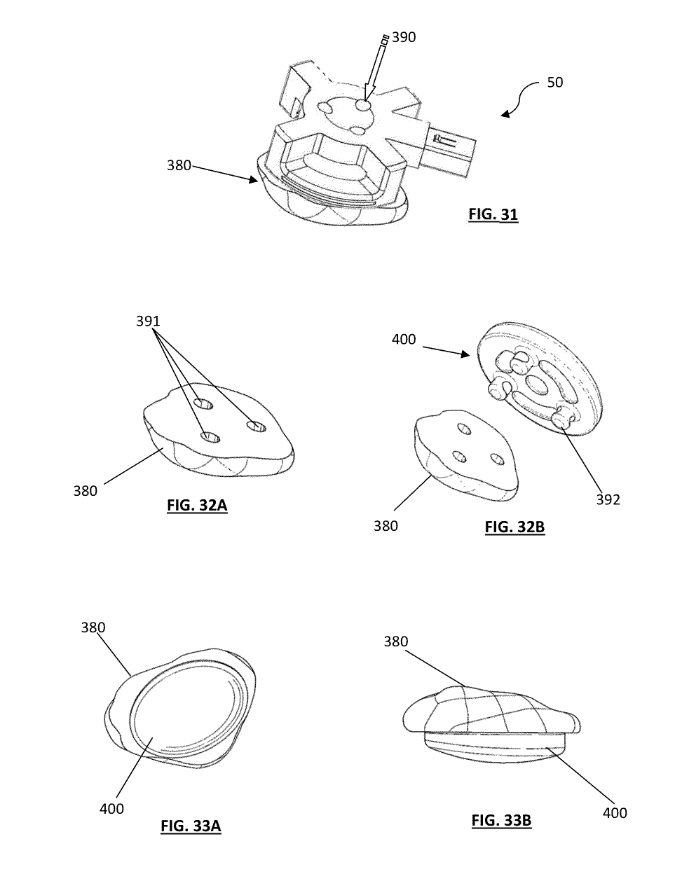

One a desired resection of the patella has been accomplished, one or more drill holes can be formed in the resected surface of the patella. As shown in FIG. 31, the patellar head 50 can include one or more drill guides 390 for the creation of drill holes in the patella 380. If desired, the drill holes can be formed prior to the resection of the patellar surface, or can be formed after resection of the patellar surface. Once the desired drill holes 391 in the patella have been formed (see FIG. 32A), a patellar button 400 can be introduced, with one or more pegs 392 that can extend into the one or more drill holes formed in the resected surface of the patella (see FIG. 32B). Once in a desired position on the patella, the button 400 can be secured to the patella 380 using bone cement or other adhesives, the pegs can press-fit into the drill holes, or various other securing mechanisms (i.e., screws, etc.) can attach the button to the patella (see FIGS. 33A and 33B).

The various patella head system embodiments disclose herein, including the multiple patellar head design, the adjustable rotatable patellar head design, and the adjustable patellar head designs, may incorporate a variety of other features and combinations of materials. For example, the various patella head systems described herein could include a measuring or assessing tool (not shown) on an opposing face to replace existing poly patella sizers in instrument kits. Such an arrangement would allow a surgeon to easily remove the patella head system from the patella clamp, reverse the head and use the opposing side to assess the size of the resected surface of the patella.

In various alternative embodiments, the various patellar head systems described herein may be uniquely designed as reversible grasping mechanisms to accommodate grasping both the patella bone and the button. If surgeon wishes to prepare the patella to resect, the surgeon may quickly connect the patella head to the patella clamp to grasp the patella. Once the patella has been resected and holes reamed to accept the button, the surgeon may insert the button onto the patella and rotate the head to orient the grasping surface towards the opposing jaw. The surgeon could then use the clamp to easily clamp the button and the patella together.

Any of the patella head system embodiments described herein may be manufactured from a variety of biomedical materials, including those that exhibit superior properties for their intended use, such as high performance polyethylenes, low friction polymers, titanium, stainless steel, or a hybrid of other biomaterial combinations thereof. The various pieces, features and/or functionalities of the illustrated patellar head systems discussed may be interchangeable, be made modular, or may be integrated across the various embodiments discussed above.

Patellar Reamer Clamp Assembly

In one alternative embodiment, a patellar reamer clamp assembly can be designed and/or selected to accommodate both standard patella shapes and/or non-standard patellar shapes, including oblong or other irregular shapes. As shown in FIG. 3, various patellar shapes may be more elongated based on the physiology of the patient or the progression of the disease, and it can be advantageous to for the surgeon to have an adaptable patellar reamer clamp assembly to accommodate a wide variation in the native shape of the patella prior to resection and drilling of holes.

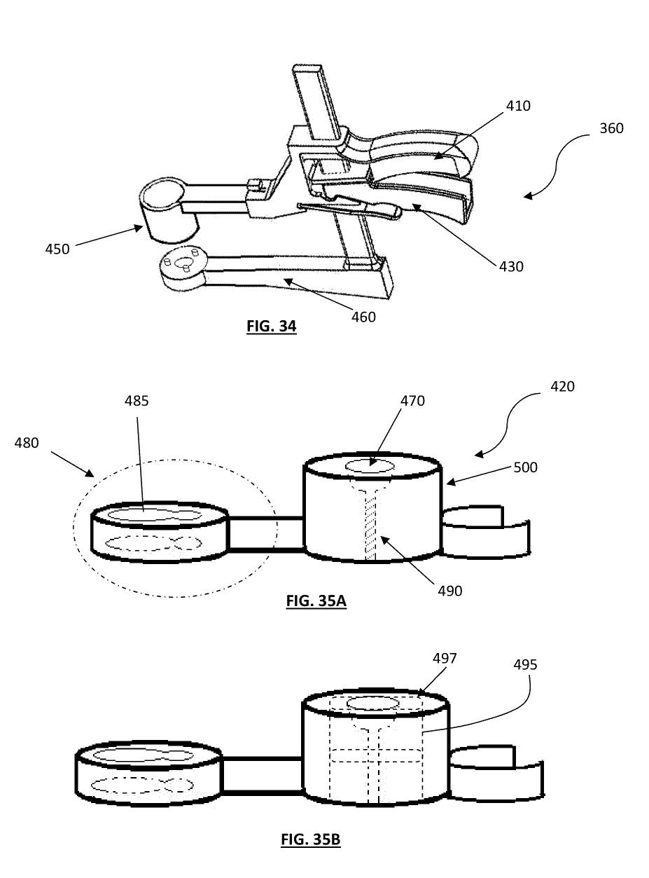

FIG. 34 depicts a side view of one alternative embodiment of a patellar reamer clamp assembly 360, illustrating a patellar base plate 460, a patellar hub 450, a handle 410, and an actuating lever 430. The patellar reamer clamp assembly and its respective parts may be manufactured from a variety of biomedical materials, including those that exhibit superior properties for their intended use, such as high performance polyethylenes, low friction polymers, titanium, stainless steel, or a hybrid of other biomaterial combinations thereof.

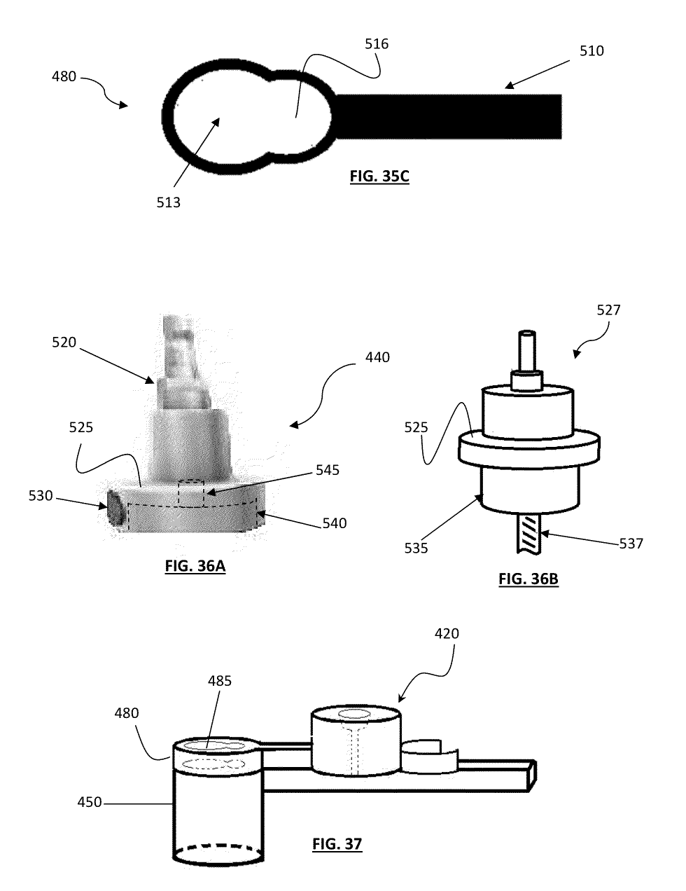

FIG. 35A illustrates a side perspective view of a patellar template 420 that can be used in conjunction with the patellar reamer clamp assembly of FIG. 34 to prepare a patella to receive a patellar implant. The patellar template 420 can be formed from a unitary piece or assembled from multiple modular and/or integrated pieces, although in one preferred embodiment the template 420 can be formed from a single piece plastic blank (not shown) or manufactured using a 3-D printing process from a CAD or other electronic manufacturing file. The patellar template 420 includes an attachment body 500 and a patellar reaming jig 480. The attachment body 500 desirably functions to secure the template 420 to an arm of the patellar hub 450, such that the reaming jig 480 extends over the patella hub 450 in a desired orientation and position. As depicted, the attachment body 500 can include a counterbore 470 and/or a threaded bore 490 (if desired), which can accommodate a compression screw or other securement mechanism for attaching the template 420 to the arm of the patellar hub 450.

The reaming jig 480 can include features having a variety of thicknesses, which in various embodiments can be employed to control or limit resection depths of drilling or reaming instruments into the patellar surface, including such depths as 2 mm to 12 mm of depth. Portions of the reaming jig 480 may incorporate thicknesses that correspond to custom or patient-specific resection depths designed specifically for the patient from pre-operative image data.

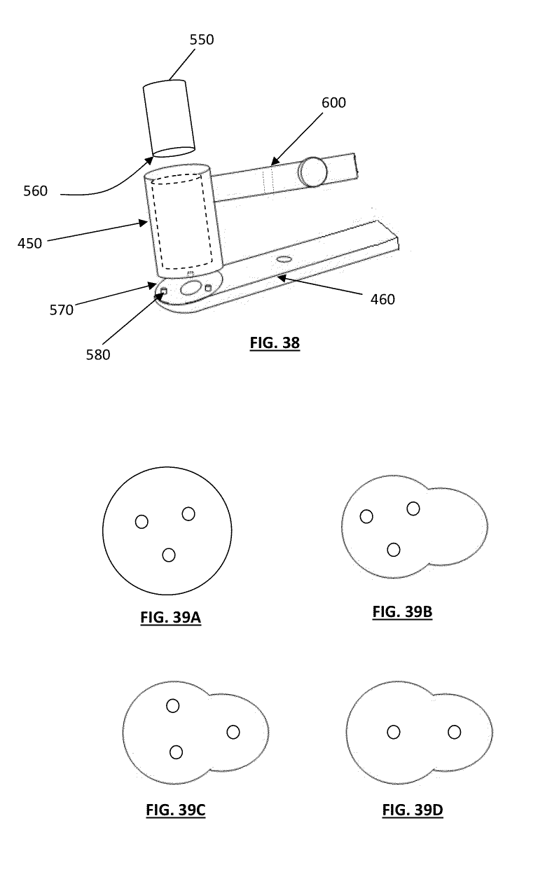

FIG. 35B illustrates a side perspective view one alternative embodiment of a patellar template 420. This alternate embodiment desirably facilitates the horizontal translation of the patellar template 420 in a predetermined manner along the arm of the patellar hub 450, where desired. The attachment body 500 includes a channel 495 that is milled through the center of the body to substantially match a securement screw diameter (not shown). If desired, a larger channel 497 may be milled into the upper surface to a certain depth to accommodate and increased diameter. In various alternative embodiments, a corresponding securement screw could include threads or other features on a lower half of the screw body, so as to thread and tighten into an opening 600 in the arm of the patellar hub 450 (see FIG. 38).

As previously noted, the reaming jig 480 of the patellar template 420 can include an oblong shaped opening 485 (see FIG. 35A) that is desirably positioned above the patellar hub 450. The oblong shaped opening may be designed and/or selected using standard oblong patellar sizes, using a library database of patella sizes, or the shape and/or size of the opening may be derived from patient-specific image data.

FIG. 37 depicts a side view of a patellar template 420 secured to an arm of the patellar hub 450. In this position, the reaming jig 480 covers the patella hub 450 in a desired orientation and position, with the opening 485 in communication with a hollow bore of the patella hub 450. In use, the oblong shaped opening 485 can desirably act as a reamer guide to allow reaming of pertinent surface portions of the patella (i.e., using one or more milling fixtures) and/or it can be used as a guide to drill specific holes into the patella or resected surface thereof. Desirably, a particularly sized reamer can be advanced into a correspondingly-sized portion of the opening 485, with the opening desirably guiding the reamer into contact with the underlying patellar surface until a desired depth stop is reached.

FIG. 35C depicts an enlarged partial top plan view of one exemplary reaming jig 480, which can be designed to accommodate a variety of sizes of reamers, including such reamer sizes as 26 mm through 38 mm in diameter. In the disclosed embodiment, the surgeon can select two reamers of differing sizes to prepare the bone, which when associated with a custom elongated patellar component, can optimize the patellar coverage in a desired manner. In various alternative embodiments, the reaming jig 480 could be formed as a modular piece with a quick-connect handle 510, that could be used to attach to a handle of the patella clamp or other feature. Such a quick-connect handle may be attached using a variety of methods or mechanisms known in the industry.

FIG. 36A depicts a side view of one embodiment of a reamer assembly 440 for use with various embodiments described herein. The reamer assembly 440 may include an integrated reamer stem 520, a lid 525, a locking mechanism 530, a hub housing counter-sink or counter-bore and a reamer countersink or counter bore 545. The reamer stem 520 can be designed to fit commonly available surgical drill chucks or reamer chucks or the manufacturer may decide to provide a custom drill or reamer the reamer stem 520. The reamer assembly 440 may also be designed to fit over or cap the patellar hub 450 and reaming jig 480, depending on the desired use during surgery. A counterbore or countersink 540 may be designed into the cap 440 and secured tightly by a locking mechanism 520 when placed over the reaming jig 480. In addition, when ready to ream or drill using a first reamer sized to approximate the large oblong patella guide hole 513 (see FIG. 35C), the stem of the first reamer can fit within the reamer countersink or counterbore 545 by a press-fit, collet or quick release mechanism. Such a releasable arrangement for securing the reamer can be advantageous to allow replacement using a variety of drill bits or reamers (not shown). When ready to begin reaming the second surface of the oblong patella, the reamer assembly 440 can be removed, the second smaller reamer 516 can replace the first larger reamer 513, and the reamer assembly can be slid towards the smaller template guide hole 516 to complete the reaming operation. These steps can be repeated to drill appropriate placement holes for posts of the patellar button, if necessary.

FIG. 36B depicts a side view of one alternate embodiment of a reamer assembly 527. This specific embodiment utilizes a reamer assembly 527 sized for each of the large template guide hole 513 and the small template guide hole 516 (see FIG. 35C). The surgeon will desirably have two reamer assemblies (one large and one small) for reaming the patella. The reamer assembly lid 525 will be designed with a cylindrical plug or base 535 that substantially matches and fits into the diameter of the large guide hole 513 or the small guide hole 516, respectively, to stabilize and guide the reaming tools surface during the drilling and reaming operation. In various embodiments, a lower surface of the plug 535 will incorporate a cutting or reaming surface, as is known in the art. Desirably, this arrangement prevents significant horizontal sliding of the respective reamer bits during the cutting operation. In various embodiments, the reamer may include an auxiliary reaming drill bit 537 for forming drill holes in the patella for accommodating corresponding pegs (not shown). The drill bit may come integrated within the reamer assembly 527, or it may be removable from the reamer and incorporate a press-fit, collet or quick release mechanism to allows quick and easily replacement of a variety of drill bits or associated cutting heads (not shown).

FIG. 38 illustrates a partial perspective view of a patellar hub 450 with an associated patellar base plate 460, and a patient specific alignment insert 550. The patellar hub 450 is desirably designed to have multiple purposes—it houses the patient specific alignment insert 550 which helps to align the patella in a desired manner, and it secures relevant portions of the patella in a desired orientation and position during the reaming operation. Desirably, the patellar hub will be sized and positioned such that at least two opposing portions of the lower rim of the hub will relatively evenly contact the patellar surface when the patella is properly positioned within the clamp (thereby securing the patella in contact with the hub). The patellar hub 450 may have patient-specific features on its lower surface if desired, or it may be relatively generic in shape (i.e., cylindrical) with small spikes or other securing features that are not patient specific. In use, the patient specific alignment insert 550, which includes one or more patient-specific features on a lower surface, can be placed inside the hub 450, such that the contact surface 560 of the insert 550 faces the patella in a desired manner and the patella is properly positioned to allow the requisite contact with the lower surface of the hub. The insert 550 may be secured within the hub using a securing mechanism such as an integrated collet within the patellar hub 450, or any other accepted securing mechanism may be designed, including the use of corresponding thread forms (not shown) and a rotation handle (not shown) on the upper surface of the insert 550.

The patellar baseplate may optionally include a patient specific patellar tray 570 with frictional elements to assist with grasping the patella, such as friction pegs 580 or one or more spikes, roughened surfaces, spikes with serrated tips, etc. The friction pegs 580 may assist with gripping the patella, and can advantageous to help secure the patella from moving while the patellar surface is being reamed. The patellar tray contact surface may also be designed to match or substantially match the contours of the facing surface of the patella.

Once the insert is secured within the hub, the clamp can then be closed around the patella, with the contact surface 560 conforming to, matching or substantially matching the corresponding surface of the patella. The surgeon may maintain a desired pressure on the patella for the remainder of the operation by squeezing the handle tightly (or the clamp may lock without further pressure from the surgeon's hands), or a standard locking or spring loading arrangement can be used to keep sufficient pressure, such as a light compressive load, on the patella. In the disclosed embodiment, once the surgeon accesses or grasps the patella, the surgeon may use the clamp as previously described to firmly position and lock the patella between the patellar hub 450 and the patellar base plate 460.

After the patella is grasped, the surgeon may remove the insert 550, thereby exposing the patella surface for the reaming and drilling process. Desirably, the lower surfaces of the patellar hub 450 (which may include gripping features and/or spikes, as previously described) and the opposing surfaces of the patellar base plate 460 will secure the patella in a desired manner and prevent relative movement and/or rotation of the patella in an undesirable fashion during the reaming and drilling operations. The patellar template 420 is then secured over the patellar hub 450, using a compression screw or other securement feature. The drill or reamer, attached to the reamer assembly, is then advanced into the opening 485 of the reaming jig 480 and the first large diameter surface will be reamed. A second sized-reamer can be selected and used to ream the second smaller diameter surface.

In various alternative embodiments, the patellar hub 450 may incorporate patient specific features, potentially eliminating a need for a separate patient specific insert 550. The patient specific-features of the hub may be integrated into the lower contact surface of the hub, and still serve as a housing and guide for the drill bit/reamer used to ream the patella surface and/or place drill holes. The patellar hub 450 may also be a modular component, which can be attached or detached using a quick release mechanism should the surgeon be interested in using other tools that can be attached to the clamp.

The clamp may incorporate a wide variety of connection features, including compressive or tensile coil springs, linear actuating slides, spring-tempered leaf springs, pivoting connections or other arrangements. The handle may include ergonomic features designed to fit common or standard hand sizes for easier grasping, or to provide for one-handed operation. In various embodiments, it may be advantageous to construct the entire adjustable-patellar reamer assembly from modular components, which may facilitate the sterilization process.

FIGS. 39A through 39D depicts various combinations of drilled holes that could be achieved using the adjustable patellar reamer clamp assembly as described herein. The placement of such drill holes can be particularized for a specific patient, or can be adapted to patellas of standard sizes and standard hole placement.

FIGS. 40A and 40B show side perspective and top plan views of one exemplary embodiment of reamed holes that may be formed into a patella using the patellar reamer clamp assembly of FIG. 34. The dimension of these holes will desirably be predetermined using patient specific data. The patellar reamer clamp assembly may be used to create an inset oblong shape 630 or counter-bore oblong shape, which can accommodate an inset patellar implant placed within the reamed region. Such an arrangement can create a peripheral wall which substantially surrounds the periphery of the implant, additionally securing the implant to the patella. In such an arrangement, additional securing pegs may be unnecessary.

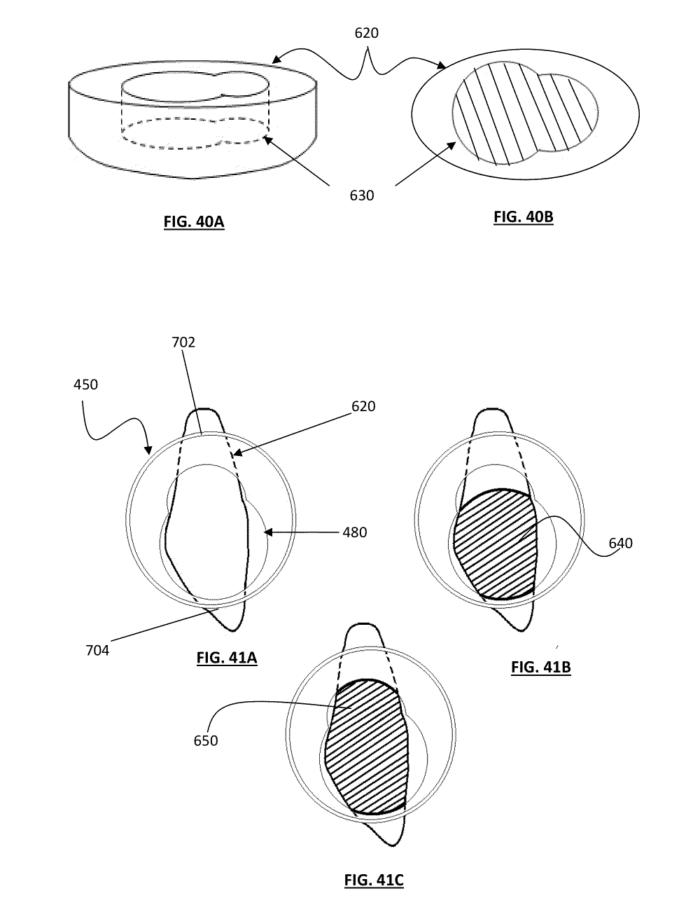

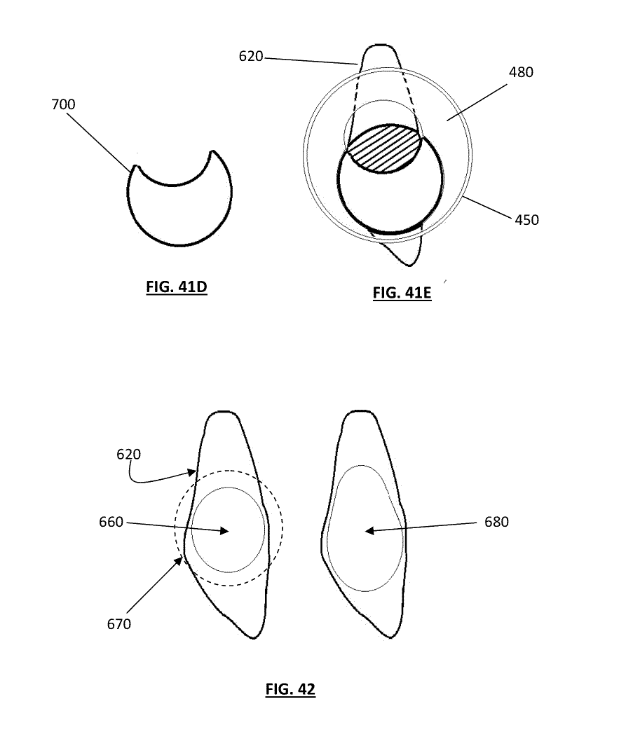

FIGS. 41A through 41C depict top plan views of the patella, reaming jig 480 and patellar hub 450, while performing various reaming operations to resect the patella in a desired manner to create a flattened or other prepared surface. FIG. 41A illustrates the surgeon's view of the patella 620, the patellar hub 450 and the reaming jig 480 prior to reaming. Initially, the surgeon will position the patellar hub 450 against the patella 620 as previously described, such that the patellar hub 450 contacts the patella in at least a first contact zone 702 and a second contact zone 704. The insert (not shown) is removed, and the reaming jig 480 is positioned over the patellar hub. The surgeon can then advance a first large diameter reamer through the reaming jig 480 and patellar hub 450 assembly, and ream the large guide hole, leaving a flattened surface 640 (shown in cross-hatch in FIG. 41B). Subsequently, the surgeon will advance a second smaller diameter reamer through the assembly and ream the small guide hole, leaving a flattened surface 650 of the patella as shown in FIG. 41C.

FIG. 41D depicts a top plan view of a reaming plug 700 formed in a partially-cylindrical or “moon” shape for use with the patellar hub 450, in which the reaming plug can be inserted against an already reamed portion of the opening to (1) confirm the first reaming operation was completed to a proper depth and extent, (2) to ensure any anchoring holes created in the reamer surface are properly positioned, and (3) to help guide the second reamer to complete reaming and preparation of the patellar surface without sliding or moving into the first reamer area. FIG. 41E illustrates the surgeon's view of the patella 620, the patellar hub 450 and the reaming jig 480, with the reaming plug 700 in position, showing the surface of the patella remaining for the second reaming operation. If desired, a second plug could be inserted to confirm the proper preparation of the remainder of the reamed surface in a similar manner.

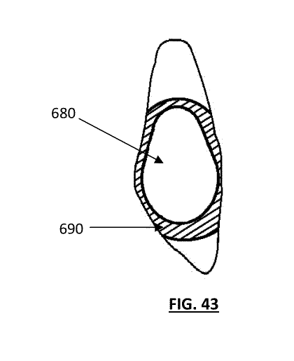

FIG. 42 illustrates a significant advantage of using the various disclosures and tools described herein. Where the patella is significantly elongated, which can occur in a significant portion of the patient population, a standard sized patellar button may be suboptimal when used for resurfacing of oblong shaped patellas 620. In such a case, once a surgeon discovers that the patient has an oblong patella 620, the surgeon will typically choose a standard sized patellar button to repair or replace the native or damaged patella. Many manufacturers provide surgeons standard sized patellas at set dimensions and shapes, generally circular or near-circular. The use of such buttons may lead to placement of an undersized patellar button 660, which gives little patellar coverage and can cause native patellar bone to be exposed and rub against the knee during flexion and extension, causing significant pain as well as potential misalignment of the normal rotation of the knee. However, similar problems may occur when the surgeon attempts to use an oversized patellar button 670. In such a case, the edges of the polyethylene button will generally contact adjacent tissues, similarly causing pain and potentially causing the alignment to be affected. Accordingly, the methods and devices described herein can be employed to ream an oblong shaped inset and/or oblong flat surface (such as shown in FIG. 36A), or ream the surface flat (such as shown in FIG. 41C or 43) to provide appropriate patellar coverage using an oblong shaped patellar button 680 to ensure that sufficient surface contact between the patellar button and the patella is obtained and the knee alignment is proper and the repair is optimized.

The use of reamers and similar tools in this manner with the patellar clamp of FIG. 34 can also facilitate the creation of curved, irregular and/or unusual geometry on the patellar surface. Curved (i.e., concave and/or convex tipped) reamers and/or other surgical preparation tools could be employed to create a convex-shaped and/or concave-shaped reamed patellar surface, which could allow for increased patellar thickness in localized areas after reaming and patellar button implantation. Where desired, the corresponding surfaces of the patellar implants can be similarly shaped to accommodate the specific patellar anatomy created during the reaming operation.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

### EQUIVALENTS

The invention may be embodied in other specific forms without departing from the spirit or essential characteristics thereof. The foregoing embodiments are therefore to be considered in all respects illustrative rather than limiting on the invention described herein. Various modifications to the embodiments described will be readily apparent to those skilled in the art, and the generic principles defined herein can be applied to other embodiments and applications without departing from the spirit and scope of the present invention as defined by the appended claims. The true scope of the invention is thus indicated by the descriptions contained herein, as well as all changes that come within the meaning and ranges of equivalency thereof, and the present invention is not intended to be limited to the embodiments shown, but is to be accorded the widest scope consistent with the principles and features disclose herein.

What is claimed is:

1. A surgical instrument for grasping a patellar bone of a patient during joint surgery, comprising:
a first grasping element having a first bone contacting surface, the first bone contacting surface having an outer surface that is shaped to substantially conform to an anatomical surface of a portion of the patellar bone;
a second grasping element having a second bone contacting surface; and
at least one of the first or second grasping elements including a guide for aligning a surgical tool to cut, drill or ream at least a portion of the patellar bone;
wherein at least one of the first and second grasping elements can be moved to grasp the patellar bone between the first and second grasping elements, such that the first bone contacting surface contacts the substantially conforming anatomical surface of the patellar bone and the second bone contacting surface contacts a generally opposing surface of the patellar bone.

2. The surgical instrument of claim 1, wherein the first grasping element is removably connected to the surgical instrument.

3. The surgical instrument of claim 1, wherein the guide is adjustable relative to the first grasping element.

4. The surgical instrument of claim 1, wherein the guide is adjustable relative to the second grasping element.

5. The surgical instrument of claim 1, wherein the second bone contacting surface is shaped to substantially conform to the generally opposing surface of the patellar bone.

6. The surgical instrument of claim 1, wherein the second grasping element is removably connected to the surgical instrument.

7. The surgical instrument of claim 1, further comprising an indicator to identify the relative distance between the first and second grasping elements.

8. The surgical instrument of claim 1, wherein both the first and second grasping elements are removably connected to the surgical instrument.

9. A surgical instrument for grasping a patellar bone of a patient during joint surgery, comprising:
a first grasping element having a first bone contacting surface;
a second grasping element having a second bone contacting surface and a third bone contacting surface, at least a portion of the third bone contacting surface being selectively removable from the second grasping element; and
the third bone contacting surface having a surface that is shaped to substantially conform to an anatomical surface of a portion of the patellar bone;
wherein at least one of the first and second grasping elements can be moved to grasp the patellar bone between the first and second grasping elements, such that the third bone contacting surface contacts the substantially conforming anatomical surface of the patellar bone.

10. The surgical instrument of claim 9, wherein the second grasping element comprises a hub having an opening formed there through and an insert sized to fit within the opening, the second bone contacting surface being located on a surface of the hub adjacent to the opening, and the third bone contacting surface being formed on the insert.

11. The surgical instrument of claim 9, wherein the second grasping element is removably connected to the surgical instrument.

12. The surgical instrument of claim 9, wherein the first grasping element is removably connected to the surgical instrument.

13. The surgical instrument of claim 9, wherein the first bone contacting surface is shaped to substantially conform to an anatomical surface of the patellar bone.

14. The surgical instrument of claim 9, wherein the second bone contacting surface includes surface features that are particularized to anatomical features of the patient.

15. The surgical instrument of claim 9, further comprising a removable patellar guide for attachment to the second grasping element, the patellar guide including at least one guiding feature for aligning a surgical tool to cut, drill or ream at least a portion of the patellar bone.

16. A method of preparing a bone surface of a patella during knee surgery, the method comprising:
determining a three-dimensional shape of a portion of the patella and producing a grasper having a first and second grasping element, at least one of the first and second grasping elements including a first bone contacting surface that conforms to the portion of the patella;
aligning the first bone contacting surface with the conforming portion of the patella;
grasping the patella with the first and second grasping elements such that the first bone contacting surface is aligned with the conforming portion of the patella; and
preparing the bone surface by cutting, drilling or reaming at least a portion of the bone surface while the patella is grasped between the first and second grasping elements.

17. The method of claim 16, wherein at least one of the first and second grasping elements further comprises a guiding feature for aligning a surgical tool to cut, drill or ream at least a portion of the patella, and the step of preparing the bone surface by cutting, drilling or reaming at least a portion of the bone surface while the patella is grasped between the first and second grasping elements comprises using the guiding feature to align a surgical instrument for preparing the bone surface by cutting, drilling or reaming at least a portion of the bone surface while the patella is grasped between the first and second grasping elements.

18. The method of claim 16, wherein the step of grasping the patella with the first and second grasping elements such that the first bone contacting surface is aligned with the conforming portion of the patella prevents the patella from rotating relative to the first and second grasping elements.

19. The method of claim 16, where the step of preparing the bone surface by cutting, drilling or reaming at least a portion of the bone surface while the patella is grasped between the first and second grasping elements comprises preparing the bone surface by cutting or reaming at least a portion of the bone surface to a generally planar shape while the patella is grasped between the first and second grasping elements.

20. The method of claim 16, where the step of preparing the bone surface by cutting, drilling or reaming at least a portion of the bone surface while the patella is grasped between the first and second grasping elements comprises preparing the bone surface by drilling or reaming at least a portion of the bone surface to an inset shape while the patella is grasped between the first and second grasping elements.

21. The method of claim 16, further comprising the step of removing the first bone contacting surface from the first or second grasping element while the patella is grasped between the first and second grasping elements immediately before the step of preparing the bone surface by cutting, drilling or reaming at least a portion of the bone surface while the patella is grasped between the first and second grasping elements.
